(19) United States
(12) Patent Application Publication
(10) Pub. No.: US 2022/0211522 A1
(43) Pub. Date: Jul. 7, 2022

(54) 3D PRINTED PROSTHETIC LINERS AND SOCKETS

(71) Applicant: OTTO BOCK HEALTHCARE LP, Austin, TX (US)

(72) Inventors: Charles C. POLTA, Cottonwood Heights, UT (US); Florian Wihelm Sebastian Poser, Salt Lake City, UT (US); Vaughn Roy Anderson, Highland, UT (US); Douglas Rush, Salt Lake City, UT (US); Michael Leach, Salt Lake City, UT (US); Vijay Morampudi, Midvale, UT (US)

(73) Assignee: OTTO BOCK HEALTHCARE LP, Austin, TX (US)

(21) Appl. No.: 17/700,361

(22) Filed: Mar. 21, 2022

Related U.S. Application Data
(63) Continuation of application No. 16/687,532, filed on Nov. 18, 2019.

(51) Int. Cl. A61F 2/80; A61F 2/50; B29C 64/40; B29C 64/188; A61F 2/78

(52) U.S. Cl. CPC A61F 2/80; A61F 2/5044; A61F 2/7812; B29C 64/188; B29C 64/40

(57) ABSTRACT

A prosthetic device includes a polymer lattice structure. The lattice structure includes a first surface arranged to face toward a residual limb, a second surface arranged to face away from the residual limb, a thickness between the first and second surfaces, and a variable lattice density across the thickness.

### TECHNICAL FIELD

The present disclosure relates generally to prosthetic devices, and more particularly relates to prosthetic liners and sockets, and related methods for making prosthetic liners and sockets.

### BACKGROUND

It is highly desirable that prosthetic liners conform closely to the residual limb, and accommodate all surface contours and sub-surface bone elements of the residual limb. They should also provide a comfortable cushion between the residual limb and the hard socket of the prosthesis that is to be fitted over the residual limb.

Generally, liners are made from silicone, polyurethane or other elastomeric materials that have been formulated as suitable substances for suspension type liners. Such elastomer materials are configured to have the appropriate hardness, elongation, tensile, and other properties, to provide a comfortable, as well as a functional liner.

Much like prosthetic liners, orthotic or prosthetic sleeves provide support and reinforcement for muscles, joints, and extremities of those in need of assistance, such sleeves are not limited to use for amputees but may be applied to existing limbs to provide support in a manner associated with conventional orthotic devices. Orthotic and prosthetic sleeves of this type are described in, for example, U.S. Pat. No. 6,592,539.

While effective solutions have been proposed and implemented, it is still highly desirable to improve comfort of such liners or sleeves to ever so increase their ability to conform to irregularities on a residual limb, to accommodate a wider variety of limbs with fewer sizes of liners, and provide an amputee with enhanced comfort at a residual limb interface with a prosthesis while maintaining sufficient strength and durability. Moreover, it is particularly desirable to provide a liner or sleeve wherein means is made available which distributes pressure of the liner against a prosthesis while providing superior stretchability.

Prosthetic limbs are attached to a human residual limb by various suspension means. A sleeve is a common method of suspension. A sleeve is a tubular structure with an opening at each end and is typically constructed of the same or similar materials used in liners. A sleeve overlaps the socket, the proximal portion of the liner which extends above the socket, and a portion of the amputee's leg. A sleeve increases the amount of skin contact between a prosthetic leg and the residual limb and also creates a hermetic seal between the limb and the socket. Additional types or methods of suspension may be used in addition to or in place of a sleeve, such as a pin on the distal end of the liner which is retained by a mechanism located at the distal end of a socket. Suction suspension utilizes a one way valve where the valve is located in the socket wall or pneumatically connected to the socket by a fitting or tubing. When the liner residual limb is inserted into the socket, air is expelled and in combination with a sleeve, a low vacuum condition is created between the liner and the socket. The resulting vacuum condition creates suspension forces which retain the prosthetic leg onto the limb. Active vacuum suspension employs a pump which creates a high vacuum condition between the liner and the limb and essentially eliminates all motion, or pistoning, between the liner and socket. All suspension methods rely on friction between the liner, sleeve (if utilized) and the residual limb to retain the prosthetic leg on the residual limb.

Because the polymeric material of the liner grips the skin of the residual limb, the socket and associated prosthesis is retained on the residual limb. However, the inherent nature of the liner material not only grips tightly on the residual limb, it also insulates the limb, trapping moisture and heat. If a sufficient amount of perspiration is trapped between the residual limb and the liner interior, then the interface between the skin and the liner and sleeve becomes lubricated by sweat and the grip of the liner on the residual limb is reduced and suspension of the prostheses may be compromised. Virtually all amputees experience significant discomfort due to the lack of ventilation and the insulatory nature of prosthetic socket environment. This environment creates several undesirable conditions, i.e.: (1) elevated skin temperature in combination with moisture results in decreased tissue strength and increased susceptibility to tissue damage, which makes the skin more susceptible to rashes, ulcers, and discomfort; (2) the moist, warm skin environment creates conditions conducive for bacterial growth that makes the skin susceptible to bacterial infection and other skin disorders; and (3) the trapped sweat results in a foul odor emanating from the limb.

One primary purpose of a prosthetic liner is to improve the pressure distribution between the limb and the socket. Prosthetic sockets are custom made devices, however; existing construction processes are not perfect and internal socket shapes don't match limb shapes perfectly. In addition, residual limbs are highly variable with regards to how much tissue is covering bony structures. Some regions of a residual limb are all soft tissue with no underlying bone structure and some areas have only skin covering the bone structure. Therefore a great deal of variation exists in the amount of cushion between the bone and a socket, and hence bony areas with little tissue between the bone and socket experience high pressures while areas with significant amounts of soft tissue between the bone and socket experience low pressures. High interfacial pressures between the residual limb and the socket result in tissue damage and discomfort.

Current prosthetic sockets are typically made of rigid carbon fiber composite materials. Temporary sockets, frequently referred to as check sockets, are constructed of thick thermoplastic materials and are also rigid. Sockets are typically formed as rigid structures to facilitate the transfer forces from the residual human limb to a prosthetic device, such as a prosthetic foot located proximal of the socket. The purpose of the socket and liner, which are frequently used in combination with a sleeve, is to secure and attach a prosthetic limb to a user's residual limb. Sufficient socket rigidity is required so the proximal prosthetic device is reliably located in space, for example, to facilitate ambulation. Due to various limitations of current socket manufacturing processes, a socket is largely monolithic. Minor variations in the thickness and the layering of fiber reinforcement layers are possible and utilized. However, the end result is that a prosthetic socket is rigid and it is not practical to use a socket without a liner due to discomfort between the residual limb and the socket. Furthermore, existing prosthetic sockets, liners, and sleeves provide insufficient cooling the residual human limb.

Liners for orthotic devices function in a similar manner to prosthetic liners, with a soft surface against the skin and a harder backing layer against the soft layer. Because a orthotic device transfers less force to the human body, softer and less expensive materials may be used. An orthotic liner typically includes fabric layered secured to a foam material, where the fabric is arranged to contact the skin of the user. The foam material of an orthotic liner is typically layered against a thermoplastic material and/or a strap. When combined, the layers of fabric, foam, and a thermoplastic and/or strap constitute an orthotic support. Fabric and foam materials absorb moisture and sweat in addition to providing limited friction against the skin. Providing optimal friction against skin is a useful way of transferring forces and maintaining an optimal location of an orthotic or prosthetic device on the human body.

For the foregoing reasons, there is a need to provide improved liners, sleeves and sockets that provide improved fit, conformability, and pressure distribution. There also is a need to provide these components with improved air circulation and increased heat conduction characteristics

### SUMMARY

One aspect of the present disclosure relates to a prosthetic device that includes a polymer lattice structure. The lattice structure includes a first surface arranged to face toward a residual limb, a second surface arranged to face away from the residual limb, a thickness between the first and second surfaces, and a variable lattice density across the thickness.

The prosthetic device may include a flexible liner, and the lattice density increases from the first toward the second surfaces. The lattice structure may have a continuous, single-piece construction. The lattice structure may have a void content of at least 5%. The lattice structure may have a porosity that permits airflow through the thickness from the first surface to the second surface. The lattice structure may include an elastomeric material. The lattice structure may include an antimicrobial material. The prosthetic device may also include a fabric material positioned on the first surface, the lattice structure being formed directly on the fabric material.

Another aspect of the present disclosure relates to a prosthetic liner or sleeve that includes a polymer lattice structure having a first surface arranged to face toward a residual limb, a second surface arranged to face away from the residual limb, a porosity that permits airflow through the lattice structure from the first surface to the second surface, a closed distal end, and an open proximal end.

The lattice structure may include a thickness between the first and second surfaces, and a variable lattice density across the thickness. The lattice density may be lowest at the first surface and highest at the second surface. The lattice structure may include an elastomeric material. The prosthetic liner may include a receiver formed in the closed distal end, wherein the receiver is configured to connect the liner to a prosthetic device.

Another aspect of the present disclosure relates to a prosthetic sleeve. Prosthetic sleeves are commonly used to create a seal between a socket, a liner, and a residual limb such that a negative pressure environment can exist within the socket. Prosthetic sleeves may also be used as a frictional suspension system by providing friction between the skin of the residual limb and the sleeve, and friction between the sleeve and the liner and/or socket, thus keeping a prosthetic limb attached to the residual limb.

A further aspect of the present disclosure relates to a prosthetic socket that includes a first surface arranged to face toward a residual limb, the first surface having a first rigidity, a second surface arranged to face away from the residual limb, the second surface having a second rigidity that is greater than the first rigidity, and a continuous, single-piece construction.

The first surface may include a first structural density and the second surface includes a second structural density. The prosthetic socket may also include a plurality of apertures formed therein and extending through at least the second surface. The prosthetic socket may also include a closed distal end, an open proximal end, a hollow interior sized to receive the residual limb, and connection feature formed in the closed distal end.

The present disclosure also is directed to a method of manufacturing a prosthetic device. The method may include forming a first portion of the prosthetic device with a first lattice structure, and forming a second portion of the prosthetic device with a second lattice structure having at least one of a different property than that of the first lattice structure. The first and second lattice structures are formed as a continuous, integral structure using an additive manufacturing process.

The at least one different property may include at least one of lattice density, material composition, lattice structure, compressibility, porosity and rigidity. The first portion may be a liner and the second portion may be a socket. The prosthetic device may be a flexible liner, the first portion may be an inner layer of the liner, and the second portion may be a second layer of the liner. The prosthetic device may be a socket, the first portion may be a first layer of the socket, and the second portion may be a second layer of the socket.

The foregoing has outlined rather broadly the features and technical advantages of examples according to the disclosure in order that the detailed description that follows may be better understood. Additional features and advantages will be described hereinafter. The conception and specific examples disclosed may be readily utilized as a basis for modifying or designing other structures for carrying out the same purposes of the present disclosure. Such equivalent constructions do not depart from the spirit and scope of the appended claims. Features which are believed to be characteristic of the concepts disclosed herein, both as to their organization and method of operation, together with associated advantages will be better understood from the following description when considered in connection with the accompanying figures. Each of the figures is provided for the purpose of illustration and description only, and not as a definition of the limits of the claims.

### BRIEF DESCRIPTION OF THE DRAWINGS

A further understanding of the nature and advantages of the embodiments may be realized by reference to the following drawings. In the appended figures, similar components or features may have the same reference label.

FIG. 1 is a cross-sectional side view of an example 3D printed prosthetic device in accordance with the present disclosure.

FIG. 2 is a cross-sectional side view of another example 3D printed prosthetic device in accordance with the present disclosure.

FIG. 3 is a cross-sectional side view of another example 3D printed prosthetic device in accordance with the present disclosure.

FIG. 4 is a cross-sectional side view of another example 3D printed prosthetic device in accordance with the present disclosure.

FIG. 5 is a cross-sectional side view of another example 3D printed prosthetic device in accordance with the present disclosure.

FIG. 6A is a side view of another example 3D printed prosthetic device in accordance with the present disclosure.

FIG. 6B is a close up view of a cross-section of the 3D printed prosthetic device shown in FIG. 6A.

FIG. 7 is a close up view of a cross-section of another example 3D printed prosthetic device in accordance with the present disclosure.

FIG. 8 is a close up view of a cross-section of another example 3D printed prosthetic device in accordance with the present disclosure.

FIG. 9 is a cross-sectional side view of another example 3D printed prosthetic device in accordance with the present disclosure.

FIG. 10 is a perspective view of another example 3D printed prosthetic device in accordance with the present disclosure.

FIG. 11 is a perspective view of another example 3D printed prosthetic device in accordance with the present disclosure.

FIG. 12 is a cross-sectional side view of another example 3D printed prosthetic device in accordance with the present disclosure.

FIG. 13. is a cross-sectional side view of a distal end portion of another example 3D printed prosthetic device with a locking pin in accordance with the present disclosure.

FIG. 14 is a cross-sectional side view of a distal end portion of another example 3D printed prosthetic device with a locking pin in accordance with the present disclosure.

FIG. 15 is a chart showing atomic lattice structures for use with the 3D printed prosthetic devices of the present disclosure.

FIGS. 16A-16F are perspective views of example lattice structures for use with the 3D printed prosthetic devices disclosed herein.

FIG. 17 is a flow diagram illustrating an example method in accordance with the present disclosure.

While the embodiments described herein are susceptible to various modifications and alternative forms, specific embodiments have been shown by way of example in the drawings and will be described in detail herein. However, the exemplary embodiments described herein are not intended to be limited to the particular forms disclosed. Rather, the instant disclosure covers all modifications, equivalents, and alternatives falling within the scope of the appended claims.

### DETAILED DESCRIPTION

The present disclosure is generally directed to prosthetic devices, and more particularly relates to prosthetic liners and sockets, and related methods for making prosthetic liners and sockets. The prosthetic liners and sockets disclosed herein may be formed concurrently and together as a unitary, integral structure. In some embodiments, the prosthetic liner, socket, or combination thereof may be formed using an additive manufacturing process, such as a 3D printing process. Various materials may be used to form the prosthetic liner and socket devices disclosed herein. In some embodiments, the same material may be used to form both the liner and socket, and a lattice structure of the liner and socket may be different to provide different amounts of stiffness, airflow, and other properties.

Additive manufacturing, also known as 3D printing, utilizes a variety of technologies to create a structure. One technology uses focused laser energy to create chemical reactions which cure liquid polymer in a bath layer by layer. Another method extrudes melted material layer by layer. These methods all add material to a part or structure in thin layers, typically about 0.003 inches to about 0.030 inches of thickness per layer. Each layer of new material being applied bonds to the existing layer by means of the melted entanglement of polymer chains or by chemical reactions or some combination of the two.

Additive manufacturing methods have the ability to create sparse structures. These structures may be similar to a 3Dimensional truss structure where rods or beams of material are connected produce efficient, light-weight structures. By altering the angles, thickness, and/or frequency of the individual rods or beams it is possible to control the mechanical response of the resulting structure.

The additive nature of the technology may provide the ability to create complex geometry and other desirable properties in a cost effective manner. Many of these geometric shapes cannot be created using other known manufacturing method. For example, liners with lattice type structures can be optimized for performance by varying the density or the geometry of the lattice. The liner can be customized in a variety of ways by merely changing the digital 3D model used to create the liner. Each liner can be customized to meet the user's specific needs with little impact on manufacturing costs.

The most common 3D printing materials used today are polymers, which are an acceptable material for liner and socket constructions. Liners and sockets created using 3D printing can have wide temperature compatibility, variable strength and/or stiffness, and be biocompatible. Since components are constructed one thin layer at a time, normal design restrictions such as angles and contour, lattice density, surface characteristics, smoothness, and undercuts do not necessarily apply to 3D printed articles. Not only does 3D printing allow more design freedom, it also allows complete customization of designs. Current additive manufacturing technologies may be perfectly suited in many instances for producing custom liners, custom sockets, and liner/socket combinations.

An example of such customization relates to a custom liner that is specifically designed for the residual limb of the individual recipient. A 3D printed liner created using a scanned data file of the limb would improve the ability to provide a correct fit of the limb for maximum comfort. In addition to providing an accurate fit, a 3D printed liner can be designed such that the structural compression stiffness, bending stiffness, and heat conduction properties can vary continuously along any dimension. Localized areas can be made softer or harder, and/or be made more rigid or more flexible. Current liner and socket designs are generally based upon a monolithic structure and properties.

The majority of residual limbs are generally cylindrical. Some, particularly short residual limbs are conical, but for the sake of simplicity and descriptive purposes, a cylindrical coordinate system will be used. The axial or longitudinal direction runs in the direction of the long arm or leg bones (femur, humerus, tibia or radius) and runs in the distal to proximal direction (or visa-versa). The radial direction is the distance from the longitudinal axis, and as applied to sockets and liners may also be referred to as the through-the-thickness direction because it extends through the thickness of a liner and socket (except at the closed end of the cylinder, where it runs in the axial or longitudinal direction). The circumferential direction is the angular position about the longitudinal axis.

While the thickness of the material may taper somewhat from the distal to the proximal ends, circumferentially, the thickness, and hence compression and bending stiffness, is typically constant in the circumferential direction, although it is possible to produce a liner with regions of increased thickness to provide additional cushion over bony areas.

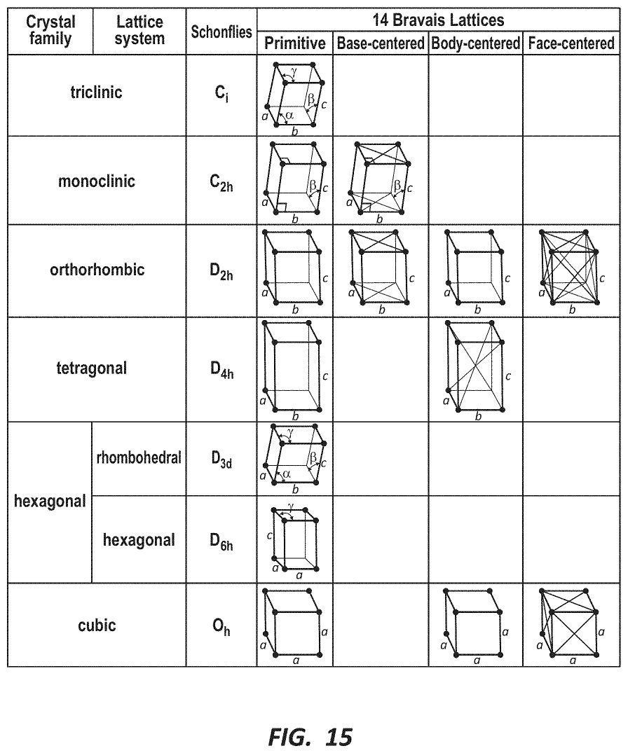

Creating a lightweight, porous, and/or variable stiffness structure by additive manufacturing techniques typically utilizes a repeating cell structure. Cell structures can mimic naturally occurring atomic structures such as cubic, tetragonal, orthorhombic, rhombohedral, monoclinic, triclinic, including body centered, face centered, and base centered variations of these atomic cells shown in FIG. 15. The atom positions in such cell structures may represent connection points between multiple individual truss members which may consists of rods or beams of material. Another naturally occurring cell structure is honeycomb. However, cell structures do not need to mimic naturally occurring structures. For example, additive manufacturing can be used to create a series of interconnected coil springs. By altering the angles, thickness, and/or frequency of the individual rods or beams used to create an additive manufactured cell structure it is possible to control the mechanical response of the resulting structure.

Various additive manufacturing technologies are available and those applicable to polymer materials include Powder Bed Fusion (PBF), Vat Photopolymerization, Material Extrusion, and Material Jetting. Powder bed fusion includes Selective Laser Melting (SLM), selective laser sintering (SLS) and selective heat sintering (SHS). PBF involves spreading a thin layer of powdered material on a surface and then melting the powder to fuse the particles. Thermal energy in the form of a laser or a heated print head provide the required melt energy.

Vat photopolymerization utilizes liquid photopolymer resin bath and a laser to create localized chemical reaction resulting in a polymer structure. Stereolithography (SLA) is the most common method with variations including Direct Light Processing (DLP) which uses microscopic mirrors to project the laser at multiple locations to eliminate the necessity of tracing each layer with the laser, and Continuous Direct Light Processing (CDLP) adds a continuously moving build platform. DLP and CDLP result in faster part build times. Vat Photopolymerization may be a preferred method to create parts made with elastomeric materials.

Material Extrusion consists of Fused Deposition Modeling (FDM) or Fused Filament Fabrication (FFF). In this process, a small thermoplastic filament is extruded through a heated nozzle and melt bonded to the previous layer of deposited material.

Material Jetting utilizes tiny print head nozzles to dispense tiny droplets of photopolymer layer by layer. UV light is used to cure the droplets. This technique is similar to the process used in ink jet printing.

Sockets are typically constructed of fiber reinforced plastic (FRP) because of the high strength, durability and low density properties of these materials. The cylindrical shape of the socket may be well-suited for the use of hard and stiff materials that do not bend or deflect significantly during use. Amputees who have been fortunate enough to retain limb joints, such as knee or elbow joints can find the relatively simple design of commonly available liners and sockets to be inadequate. For example, amputees that use below-the-knee prosthetics generally require a liner that conforms to a range of joint positions possible by an intact, functioning knee joint. As the joint moves, related tendon and muscle structures also move, and a socket must accommodate these movement, which makes creating a well-fitting socket a difficult task. Adding flexibility to a localized area of the socket can alleviate some of these difficulties.

Typical off-the-shelf liners often do not provide a comfortable fit over the entire range of motion of the joint. Even at small bending angles, the fit of the liner behind the knee can be lost due to bunching or gathering behind the knee. When flexing the knee joint, one or more folds may form in the portion of the liner overlying the region behind the knee. The folds generally occur in a lateral direction, i.e., roughly perpendicular to the length of the leg. However, more complex, crinkle-type folds can also occur. The pinching and pulling of underlying skin which can occur with such folds can result in patient discomfort. As the knee joint undergoes flexion, the relatively relaxed liner surface disposed over the kneecap (the “anterior surface’) must stretch and bend in order to accommodate the change in conformation of the knee joint, as well as the increase in anterior skin surface area which accompanies the change. A 3D printed liner could be formed in such a way to accommodate for stretch and bending by simply altering the geometry of the lattice of the polymer material.

Additionally, 3D printed structures may offer the unique ability to separate and disconnect properties which have historically been considered inherent material properties. For example, as a material is stretched in one direction/dimension the material contracts in the other two dimensions (i.e., the Poisson's effect). A 3D printed structure having a lattice structure has a response dependent on the geometry of the lattice, not necessarily on the direction a force is applied. This is different than the response of the material used to create the lattice structure. Additive manufacturing can be used to create auxetic material structures with a negative Poisson's ratio, which results in material expansion in one or more directions perpendicular to an applied tensile force and contraction in one or more directions perpendicular to an applied compressive force.

Auxetic material structures present a method to address unique problems experienced by amputees. As an amputee uses a lower limb prosthetic device over the course of a day, the pressures experienced by the residual limb result in fluid being forces out of the limb. This loss of fluid reduces limb volume and results in a poor socket fit. The most common method to deal with this volume loss is to place fabric socks over the liner during the course of the day to fill the volume in the socket. Auxetic materials have a negative Poisson's ratio. Because Auxetic materials expand in a direction perpendicular to an applied force, these structures will experience volumetric contraction when compressed, which may reduce the hydrostatic pressures in a socket and hence reduce fluid loss in the limb. Auxetic materials also experience volumetric expansion under tensile forces, which may improve the frictional forces between a liner and the skin and improve limb retention on the limb. Auxetic materials or structures may be used in combination with positive Poisson's ratio materials to create beneficial effect to an amputee.

Additive manufactured structures can be made using a variety of materials, which include thermoset polymers, thermoplastic polymers, metals, and fiber reinforced composites. Elastomers are commonly defined as rubber-like materials. Elastomers can be defined by hardness, maximum elongation, modulus, Possion's ratio, or glass transition temperature and by combinations of these properties. Elastomeric materials are available in a wide range of hardnesses or stiffnesses ranging from hard elastomers with a Young's modulus of 500,000 psi to very soft elastomers with a secant modulus of 50 psi at 100% elongation. As elastomeric materials become harder their properties become more linear elastic and follow Hooke's Law. Hence it can be difficult to differentiate an elastomer material from a non-elastomer material.

Elastomers are difficult to characterize because they have viscoelastic properties. The mechanical response of an elastomer is partially elastic and partially viscoelastic. A viscoelastic response is characterized by a linear spring in parallel with a dashpot. A dashpot is a damping device such as a hydraulic cylinder. The spring provides resistance to compression and extension, while the dashpot slows both the compression and extension, and the recovery from compression and extension. Hence the mechanical response is time dependent.

For the purposes of this application, an elastomer is defined as a material which can be stretched to 10% elongation at room temperature and recover 70% of the deformation within 10 seconds when the stretching load is removed.

Indentation hardness testing is a method of characterizing the stiffness of a material. This type of test may also be known as a durometer, indentation or durometer hardness test. Hardness testing can be performed on a wide range of materials, from the hardest steels to soft elastomers. ASTM D 2240, DIN 53505, and ISO R/868 are comparable methods used for testing both soft elastomers and hard plastic materials and the results are stated on the Shore scale. The commonly used Shore harness scales, in increasing order of hardness, are 000-S, 000, 00, 0, and A-D. The Shore D, A and 00 scales are most commonly used because these 3 scales result in a functional continuum for hardnesses in most situations. Shore-OO 63 is approximately equivalent to Shore-A 20 and Shore-A 73 is approximately equivalent to Shore-D 20. Typical commonly known material hardness are Shore-OO 20 for chewing gum, Shore-A 25 for a rubber band, Shore-A 70 for tire tread, and Shore-D 70-90 for rigid plastics like nylon and polyethylene. Some structural plastics and composite materials have hardnesses which exceed the Shore scales. Shore-000-S and Shore-000 scales are typically used for soft foams or sponges.

Most currently available prosthetic liners and sleeves have a hardness of approximately Shore-00 50, although values from around Shore-00 30 to Shore-00 70 may be in use. One of the advantages of additive manufacturing is the ability to utilize a lattice structure, sometimes known as a sparse structure. As the void content of a material increases, for example in foams, the material becomes softer. Hence, attaining the desired stiffness when utilizing a lattice structure in a prosthetic liner there may be a need to utilize a stiffer raw material, as compared to monolithic liner. The Shore hardness of the material used in an additive manufactured liner utilizing a lattice structure may extend into the Shore A or D scales for the material loaded adjacent to the skin.

When combining a liner with a socket into a single, unitary structure the stiffness of the material used may increase as the distance from the limb increases (the radial or through-the thickness direction) to provide the required stiffness and strength to resist reaction forces and yet allow a soft interface next to the residual limb. This may be achieved by changing the stiffness of the material, by changing the density of the lattice structure, by changing the geometry of the lattice, or all of the above. Therefore the stiffness of the raw material on the outside of the structure (at the maximum radial distance) may extend into the Shore-D scale and beyond.

3D printed liners and sockets provide the option of changing the lattice structure to make sections harder or softer and control the mechanical response of the structure in different directions. The cell structure can be altered such that the stiffness changes in one direction, for example, the radial or through-the-thickness direction, while leaving the stiffness in the axial and/or circumferential directions the same, or even increasing the stiffness in the axial and/or circumferential directions in different directions. With the option of changing material lattice modulus or stiffness, enhanced stability, security, fit comfort, and performance may be achieved.

In one embodiment, the material used to create a lattice structure varies in hardness through the thickness. Elastomers materials are typically available in different hardnesses and these different hardness formulations have similar atomic or chemical structures. As a result, materials from the same family product tend to bond to each other well. It is also possible to find materials from different manufacturers which bond well to each other. For example, the material used to form the liner or socket may be changed to a material of a different hardness during the additive manufacturing process. If the two materials are compatible, a strong connection will typically occur at the interface between the two materials. In one example, this approach may provide a soft inner layer against the skin and a stiffer, stronger structure on the outer layers (or visa-versa) as part of a laminated lattice structure.

In another variation, a lattice liner construction includes a trellis or web structure of polymeric material. This construction provides a porous structure, wherein air can flow freely through the liner. With the natural movements of the body, air can circulate through the socket and/or liner. This ability for air to flow through the lattice structure of the liner may provide a climatizing characteristic for improved comfort as well as enhanced skin integrity and a reduction of odor. A measure of air permeability is rate of airflow passing perpendicularly through a known area under a prescribed air pressure differential between the two surfaces of a material. A common test for fabric materials is ASTM D737-96. This test method can be adapted to thicker materials. A socket or liner, or combination of the two, with an air permeability greater than 2 ft³/min/ft² may be particularly advantageous in hot climates.

In another embodiment, the liner and socket could be made as a single piece using a 3D printing process. An inner surface of the liner (adjacent the skin of the wearer) may be relatively pliable and soft (e.g., like a cloth or even be formed directly on a sheet of cloth), and an outer surface of the liner could be made rigid so as to be supportive of the prosthesis. Both the inner and outer surfaces of the combined liner/socket structure could be made with a plurality of ventilation pathways to enhance air circulation.

Using lattice style construction for the liner and/or socket may provide passageways that improve breathability for the residual limb, thus eliminating many of the limitations of conventional liners and sockets. Further, suppleness for wearer comfort available using existing polymeric/elastomeric gel liner materials can be maintained or duplicated with a 3D printed liner structure.

Some advantages related to the 3D printed liners and sockets disclosed herein include improved breathability of the liner, socket or combination liner/socket via an open lattice structure. Such improved breathability may provide climate control, reduction of bacteria and germs, and the ability to wash the device and have near instant drying of the device. Another advantage relates to the ability to customize properties of the device. For example, the device may have a customized hardness or softness in a particular areas when using a single material. The device may have different hardness or softness in particular areas by using materials with different hardness properties. The device may have sections with different rigidity and flexibility properties. The device may have variable compressible/expandable properties that allow for physical changes in a person's residual limb. Further, as mentioned above, the socket and liner may be formed as a single-piece, unitary device. In addition, a complete prosthetic limb may be 3D printed, including a foot or hand.

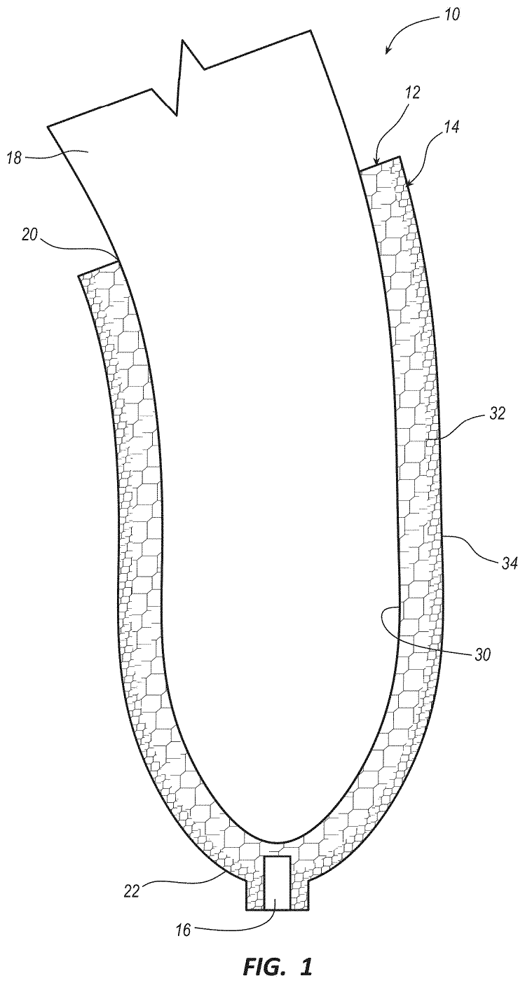

Referring now to FIG. 1, an example 3D printed device 10 is shown and described. The 3D printed device 10 includes a liner portion 12, a socket portion 14, and a receiver 16. The 3D printed device 10 is shown mounted to a limb 18, such as a residual limb remaining after an amputation. An example of a residual limb may be a residual limb associated with a below-the-knee amputation.

The liner portion 12 defines an inner surface 30. The socket portion 14 defines an outer surface 34. The inner and outer surfaces 30, 34 converge at an interface 32. The 3D printed device 10 includes a proximal end 20 (also referred to as an open end or an open proximal end), and a distal end 22 (also referred to as a closed end or a closed distal end). The 3D printed device 10 includes or defines a cavity into which the limb 18 is positioned. The receiver 16 is formed in or positioned at the distal end 22. The receiver 16 may be sized and configured for attachment of the 3D printed device 10 to a prosthetic device such as a lower leg prosthesis (e.g., a prosthetic pilon, prosthetic knee, prosthetic foot, pump mechanism, or the like). The receiver 16 is shown having a recess formed in a protruding portion at the distal end 22. Other receiver structures may be used in other embodiments, wherein the receiver may have different sizes, shapes, or orientations on the 3D printed device 10 and still provide a similar function for connection to a prosthetic device or component.

The 3D printed device 10 comprises a lattice structure. The lattice structure of the liner portion 12 is configured to have a softer, less rigid structure as compared to the lattice structure of the socket portion 14. The lattice structure of the liner and socket portions 12, 14 may have different lattice shapes, lattice sizes, and/or be comprised of different materials. In some embodiments, a solid layer may be formed along either the inner surface 30 or outer surface 34.

The solid surface may include a relatively thin layer that is formed along the lattice structure of the liner and/or socket portion 12, 14. The solid surface may provide certain advantages or properties for the 3D printed device 10. For example, a solid surface along the outer surface 34 may inhibit passage of fluid such as air, vapor and/or liquids, and/or prevents solids such as dirt and debris from passing into the lattice structure of the 3D printed device 10. A solid surface along inner surface 30 may prevent passage of fluids such as air, vapor, or liquid from passing into the lattice structure from the interior cavity of the 3D printed device 10. A solid surface along the inner surface 30 may also provide improved comfort at the interface with limb 18. In other embodiments, such as those described below, portions of the inner or outer surface 30, 34 may comprise openings to facilitate passage of air, vapor, and/or liquids into or through the wall of the 3D printed device 10 to facilitate heat transfer, humidity transfer, air circulation, cooling, heating, and the like toward or away from a limb 18 positioned within the 3D printed device 10.

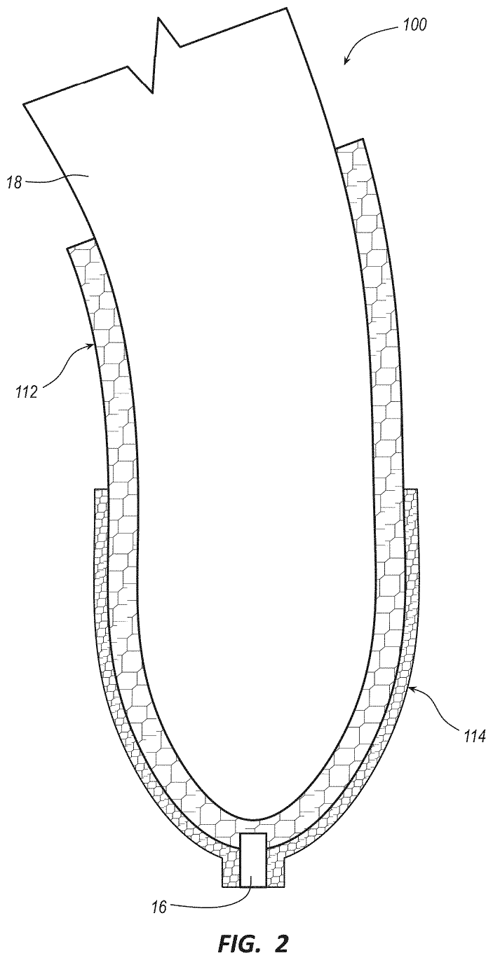

FIG. 2 illustrates another example of 3D printed device 100 having a liner portion of 112 and a socket portion 114. The socket portion 114 may be configured as a conventional rigid socket having a receiver 16 formed in a closed distal end thereof. The liner portion 112 may be formed as a relatively soft lattice structure that functions similar to a conventional liner that is used with a conventional socket. A portion of the receiver 16 may be formed in the liner portion 112.

In some embodiments, the liner portion 112 is formed separately from the socket portion 114. This separate construction may permit mounting of the liner portion 112 to the limb 18 followed by, in a later step, insertion of the limb 18 with liner portion 112 into the socket portion 114. In other arrangements, the liner portion 112 and socket portions 114 are formed as a single, unitary piece such that the liner portion 112 is inseparable from the socket portion 114. The liner portion 112 and socket portion 114 may be formed as a single continuous piece using a 3D printing manufacturing process such as those described herein.

The liner portion 112 may be formed with a lattice structure. The lattice structure may be continuous through the thickness of the sidewall of the liner portion 112. The socket portion 114 may have a lattice structure with a greater density than the density of the liner portion 112. Alternatively, the socket portion 114 may have a solid construction through its thickness.

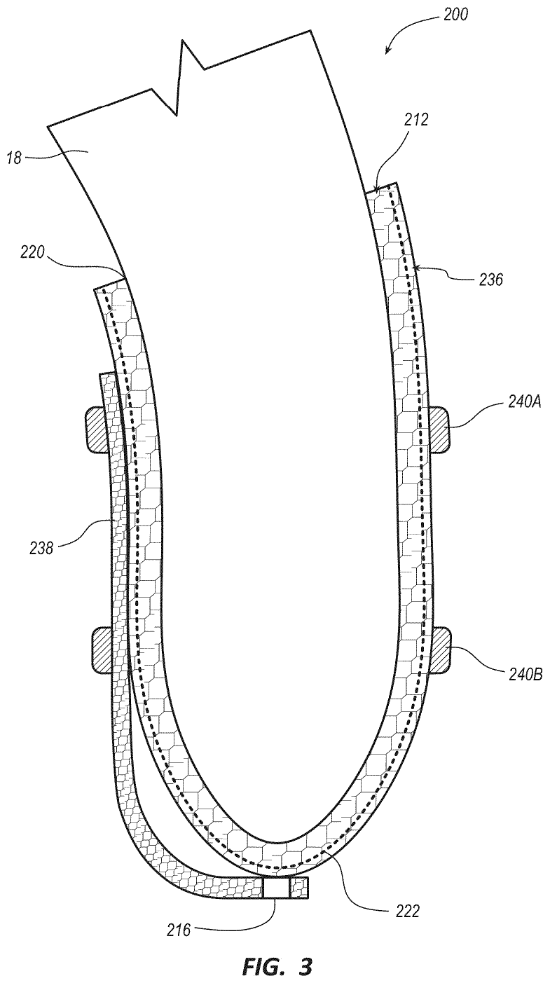

FIG. 3 illustrates another example of 3D printed device 200 mounted to a support bracket 238. The 3D printed device 200 may have a semi-rigid construction. The 3D printed device 200 may be mounted directly to the support bracket 238 to provide additional support and strength to adequately support the limb 18. The support brackets 238 may be secured to the 3D printed device 200 using a plurality of straps 240A, 240B. The straps 240A and 240B may wrap circumferentially around the exterior surface of the 3D printed device 200 and be secured directly to the support bracket 238. The straps 240A and 240B may comprise a fabric material, or may comprise a flexible polymeric material. The 240A and 240B may include brackets, clasps, fasteners or the like releasably secure or permanently connect the 3D printed device 200 to the support bracket 238. Interlocking features may be provided between the support bracket 238 and the outer shell 236 to reduce relative movement between the two parts. Straps 240A and 240B may be 3D printed and may be integrated into the support bracket 238 or the outer shell 236. Support bracket 238 may also be 3D printed.

The support bracket 238 may include a receiver 216 positioned at a distal end thereof. The receiver 216 may be aligned with a closed distal end 222 of the 3D printed device 200. In some arrangements, the 3D printed device 200 may include a recess or other receiver feature formed in the distal end 222. The receiver feature in the 3D printed device 200 may be aligned with the receiver 216 of the support bracket 238 to provide improved connection between a distal mounted prosthetic device and the 3D printed device 200 and support bracket 238.

The 3D printed device 200 may include a liner portion 212 and an outer shell 236. The outer shell 236 may have a different lattice structure than that of the liner portion 212. The liner portion 212 may have a lattice structure that provides a relatively soft interface with the limb 18. The outer shell 236 may have a semi-rigid lattice structure that provides increased rigidity as compared to that of the liner portion 212, but that typically is less than the rigidity of the socket portion 14 described above with reference to FIG. 1.

In other embodiments, the entire 3D printed device 200 may comprise a single lattice structure such as the soft lattice structure of the liner portion 212 or the semi-rigid lattice structure of the outer shell 236. In still further embodiments, the liner portion 212 and outer shell 236 may be provided along only a portion of the length of the 3D printed device 200 between a proximal open end 220 and the closed distal end 222. Various embodiments are described with reference with to the figures that follow showing different types of lattice structures at different locations along the length of the 3D printed device.

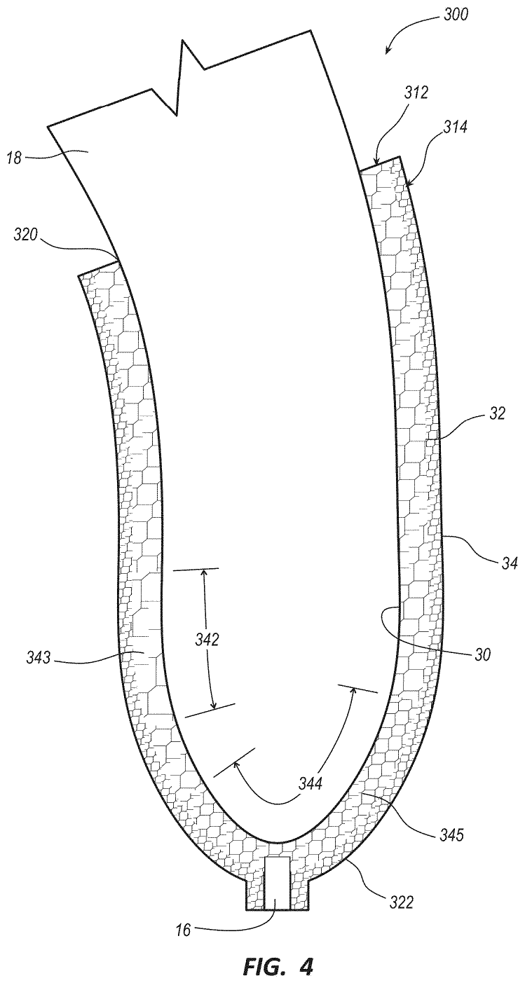

FIG. 4 illustrates a 3D printed device 300 that includes a liner portion 312, a socket portion 314, and a receiver 16. The 3D printed device 300 further includes a relatively soft area 342 and a relatively hard area 344. The soft area 342 includes a softer lattice structure 343 as compared to the lattice structure of the liner portion 312. The hard area 344 may include a semi-rigid lattice or harder lattice as compared to the lattice structure of the liner portion 312, but softer and less rigid than the lattice structure of the socket portion 314.

The soft area 342 may be positioned along a side wall of the 3D printed device 10 at a location spaced between the open proximal end 320 and the closed distal end 322. The hard area 344 may be positioned along the closed distal end 322. The soft and hard areas 342, 344 may be positioned at any desired location on the 3D printed device 300 to provide desirable properties related to the function of the 3D printed device 300, including comfort for the wearer at the interface with limb 18. For example, the soft area 342 may be positioned to interface with a portion of the limb 18 that is sensitive to pressure. The hard area 344 area may be arranged to provide additional support for the limb 18 and/or the receiver 16.

The soft and hard areas 342, 344 may extend through an entire thickness of the 3D printed device 300 from an inner surface to an outer surface thereof. Alternatively, as shown in FIG. 4, the lattice structures 343, 345 of the soft and hard areas 342, 344 may extend through only a portion of the thickness between the inner and outer surfaces, such as through the thickness of the liner portion 312 but not the socket portion 314. The 3D printed device 300 may include multiple soft and hard areas 342, 344 at any desired location. Further, the soft and hard areas may be combined or arranged side-by-side. In one embodiment, a soft area 342 is positioned within or between two hard areas 344.

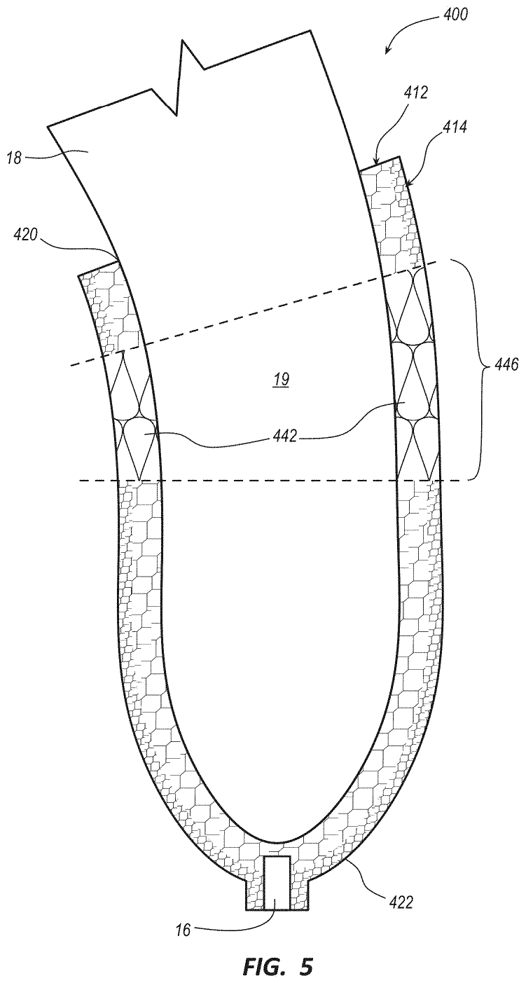

Referring now to FIG. 5, a 3D printed device 400 is shown including a liner portion 412, a second portion 414, and a receiver 16. The 3D printed device 400 also includes one or more flexible sections 446 that comprise a softer lattice 442 as compared to the lattice structures of the liner and socket portions 412, 414. The flexible sections 446 may be positioned at any desired location on the 3D printed device 400, such as along a side wall at a location spaced between an open proximal end 420 and a closed distal end 442. The soft lattice structure 442 may extend through an entire thickness of the 3D printed device between inner and outer surfaces thereof.

The flexible sections 446 may provide bending, compression, extension/expansion, and/or deformation of a 3D printed device 400 to accommodate, for example, movement of the limb 18. In one example, the limb 18 includes a joint 19, such as a knee joint, and the flexible sections 446 are aligned circumferentially with the joint 19. The flexible sections may permit, for example, compression of the 3D printed device along a back side of the joint during bending, and expansion or deformation of the 3D printed device along a front side of the joint 19 during bending. In the embodiment shown in FIG. 5, the limb 18 may include a knee joint 19 with a rear of the knee joint positioned along the left side and a front of the joint 19 positioned along the right side. The amount of flexible section 446 along the length of the 3D printed device between the proximal and distal ends 420, 422 may be greater along the front or anterior side of the joint 19 as compared to the length along the rear or posterior side of the joint 19. The flexible sections 446 may extend around only portions of a circumference of the 3D printed device 400. Alternatively, the flexible sections 446 may extend around an entire circumference of the 3D printed device 400. The flexible sections 446 may be used with any of the 3D printed device embodiments disclosed herein. Likewise, any of the features disclosed in any of the embodiments described with reference to the figures may be interchangeable with other embodiments disclosed herein.

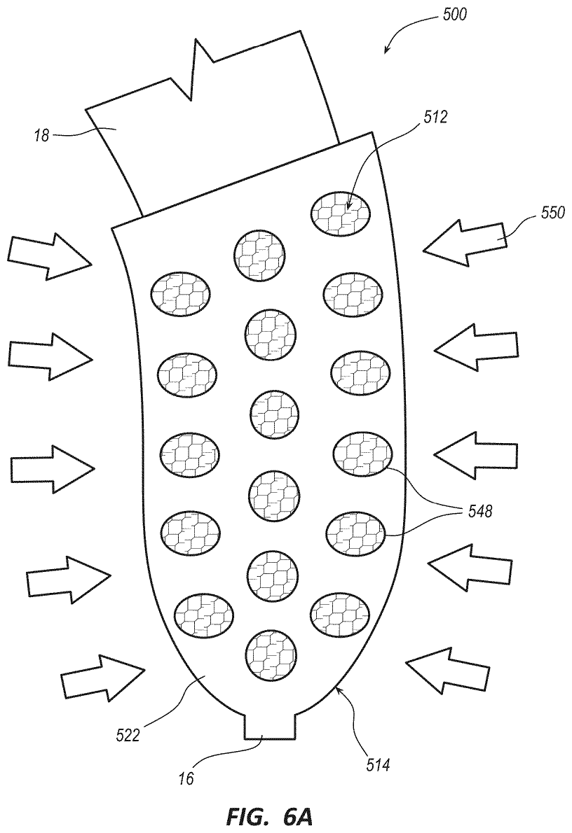

Referring now to FIGS. 6A and 6B, a 3D printed device 500 is shown including a liner portion 512, a socket portion 514, and a receiver 16. The socket portion 514 includes a plurality of ventilation ports 548 extending therethrough from the outer surface of the 3D printed device 500 to the liner portion 512 as shown in at least FIG. 6B. The liner and socket portions 512, 514 may comprise different lattice structures that provide different levels of rigidity, flexibility, airflow, compressibility, heat transfer, and other properties. The lattice structure of the liner portion 512 may permit fluid flow therethrough, such as the flow of air. The ventilation ports 548 may permit the fluid flow 550 to pass from an outer surface 534 to an inner surface 530 of the 3D printed device 500, and to permit fluid flow 550 (air, vapor and/or liquid) to pass from the inner surface 30 and the limb 18 through the thickness of the 3D printed device 500 and out through the ventilation ports 548. A fluid flow 550 may also comprise the transfer of heat to or from the limb 18. The ventilation ports 548 may also act as heat transfer ports to better facilitate transfer of heat from a limb 18 through the 3D printed device 500 to atmosphere.

The ventilation ports 548 may be positioned at any desired location along the outer surface 534 of the 3D printed device 500. In some examples, the ventilation ports 548 have a circular or oval shape, whereas in other embodiments different shapes may be used such as triangular, polygonal, elongate strips or the like. The ventilation ports 548 may be arranged in a pattern, such as rows and/or columns. In other embodiments, the ventilation ports 548 are arranged randomly along the outer surface 534. In some embodiments, one or more ventilation ports 548 may be positioned along the closed distal end 522 and act as one or more drainage ports to permit flow of liquid (e.g., perspiration) from the limb 18 out of the 3D printed device 500. The ventilation portion 548 may have different sizes at various locations on the outer surface 534. The liner portion 512 may have different lattice structures at different locations on the 3D printed device 500 to provide different amounts or types of fluid flow 550 that are intended or advantageous for that portion of the device.

The 3D printed device 500 may include a single lattice structure, the dual lattice structure shown in FIGS. 6A and 6B, or three or more lattice structures at various locations through the thickness, along a length, or around a circumference of the 3D printed device 500.

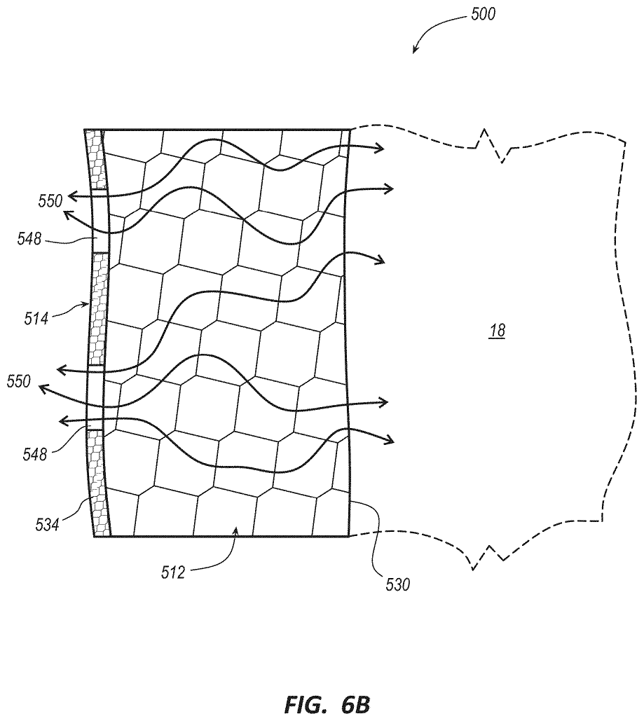

FIG. 7 shows a close up view of another example 3D printed device 600 that includes a liner portion 612 that includes a relative soft lattice structure, a socket portion 614 that includes a relatively rigid lattice structure, and an intermediate portion 636 that includes a semi-rigid lattice structure. The 3D printed device 600 may also include an inner surface layer 652 along inner surface 630 that includes a cloth-like material or a lattice structure that provides a cloth-like feel for the user. The inner surface layer 652 may comprise a lattice structure that is different from the other liner portions 612, 614, 636. In at least some embodiments, the inner surface layer 652 may comprise a different material than the materials used for the other portions 612, 614, 636. In some embodiments, the inner surface layer 652 may comprise a fabric material or fabric fibers or polymer materials typically used in fabric fibers. The inner surface layer 652 may be formed concurrently with one or more of the other portions 612, 614, 636 using a 3D printing process.

The 3D printed device 600 may be referred to as an integral socket and liner device. The 3D printed device 600 may be referred to as an integral liner with an internal fabric surface or internal surface with a fabric-like feel.

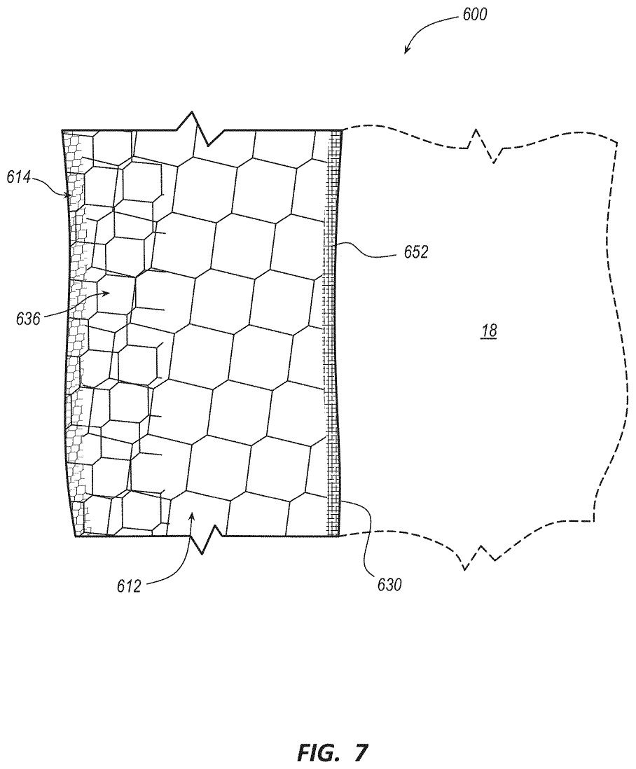

FIG. 8 illustrates a 3D printed device 700 that includes at least a liner portion 712 and an inner surface layer 752 positioned along an inner surface 730. The inner surface layer 752 may comprise the same or similar properties, attributes, lattice structure and/or material composition as described above for inner surface layer 652. The 3D printed device 700 may be referred to as a 3D printed liner having a first lattice structure associated with the liner portion 712 and a second lattice structure associated with the inner surface layer 752. The 3D printed device 700 may be used with a traditional prosthetic socket, or may be used with other devices such as the support bracket 238 described with reference to FIG. 3 or any of the other 3D printed devices disclosed herein.

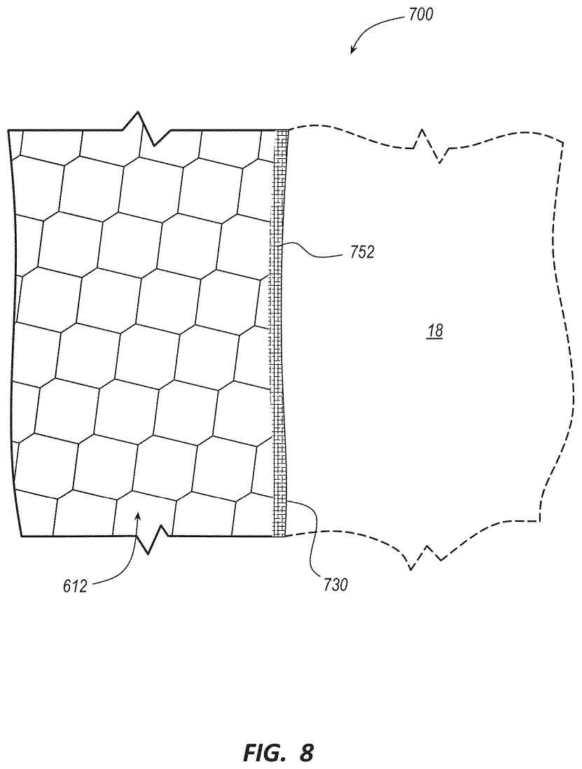

FIG. 9 illustrates another example 3D printed device 800. The 3D printed device 800 includes a liner portion, 812, a socket portion 814, and a receiver 16. The 3D printed device 800 may have a different thickness T₁ at one location (a standard thickness), a second thickness T₂ (a greater thickness) at another location, and a third thickness T₃ (a reduced thickness) at other locations along the 3D printed device 800. The different thicknesses, T₁, T₂, T₃ may be provided by varying a thickness of the liner portion 812. In other embodiments, the thicknesses T₁, T₂, T₃ may be varied by changing the thickness of the socket portion 814, or by changing the thickness of both of the liner and socket portions 812, 814. In other embodiments, an additional lattice structure or layer may be added in the area for the increased thickness T₂, which may be referred to as an increased thickness area 892. A reduced thickness portion 894 may have the thickness T₃. The remaining portions may be referred to as standard thickness areas 890, having a thickness T₁.

In some embodiments, the 3D printed device 800 may include only the standard thickness area 890 with thickness T₁ and increased thickness area 892 with thickness T₂. In other embodiments, the 3D printed device 800 may include standard thickness areas 890 having thickness T₁ and one or more reduced thickness portions 894 having thickness T₃. In further embodiments, the 3D printed device 800 may include multiple reduced thickness sections 894 and/or multiple increased thickness sections 892 and/or no standard thickness areas 890.

FIG. 9 illustrates the increased thickness area 892 having a gradually increasing thickness that varies from the standard thickness T₁ to the increased thickness T₂. The reduced thickness portion 894 is shown having a thickness that varies from the standard thickness T₁ gradually to the reduced thickness T₃. These gradual changes in the thickness may be customized to match the contours, shapes, sizes and surface features of the limb 18.

Generally, the 3D printed device 800 may have a relatively constant outer perimeter size defined by the socket portion 814 and the outer surface 834, and a variable sized or shaped inner surface 830 defined by the minor portion 812. In other embodiments, such as mentioned above, additional layers may be added, for example in the increased thickness area 892. In the reduced thickness areas 894, the thickness of both the liner portion 812 and socket portion 814 may be reduced, or only the thickness of the liner portion 812 may be reduced. As with the other embodiments disclosed herein, more than two different lattice structures may be used through the thickness of the 3D printed device, or only one of the lattice structures (e.g., the relatively hard or rigid lattice structure of the socket portion 814) may be used at certain locations such as adjacent to the receiver 16 at the closed distal end 822 or only the relatively soft lattice structure of the liner portion 812 in the area of the open proximal end 820.

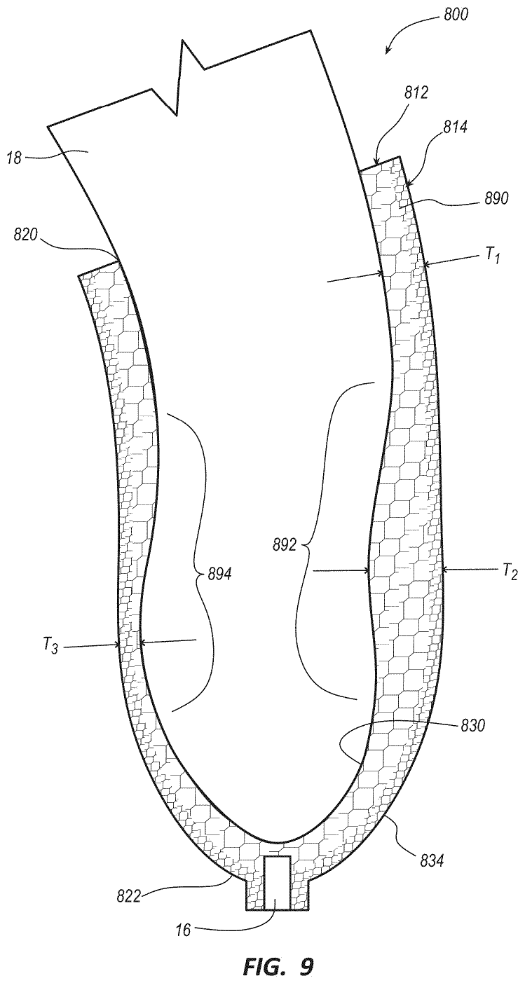

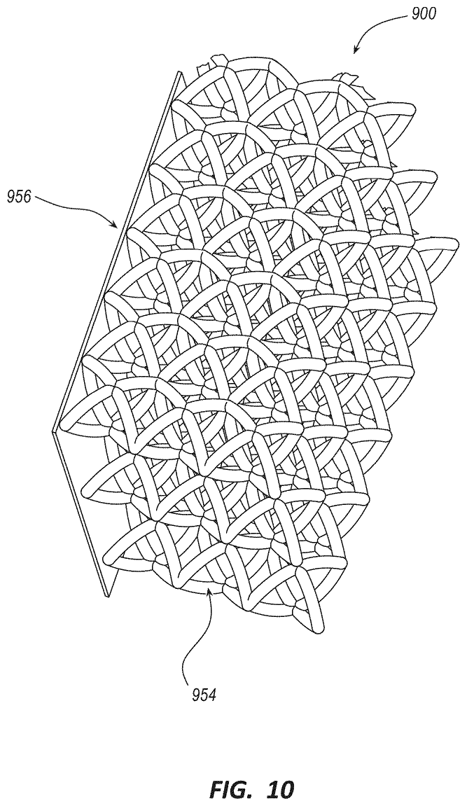

FIGS. 10 and 11 illustrate example lattice structures that may be used with the various 3D printed devices disclosed herein. FIG. 10 illustrates a 3D printed device 900 that includes an outer layer 956 that is substantially solid and/or continuous, and a first lattice structure 954 that is relatively open and comprised of substantially linear crossing members or struts. FIG. 11 shows a second lattice structure 1054 and an outer layer 1056. The outer layer 1056 may be substantially solid and/or continuous rather than including an open lattice structure. The second lattice structure 1054 may have different shaped and sized struts, such as struts that have a contoured shape, and the resulting lattice structure may include circular, spherical or other shapes.

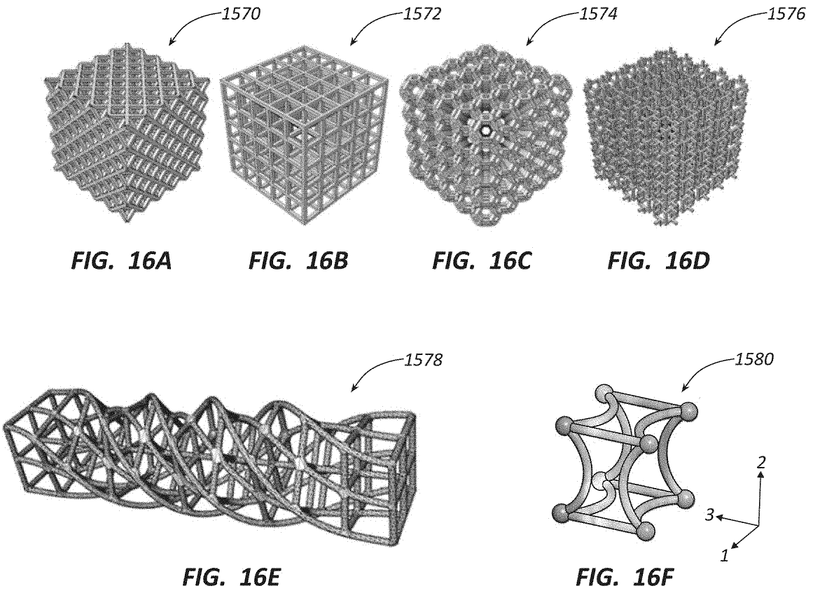

The first and second lattice structures 954, 1054 shown in FIGS. 10 and 11 are exemplary only. Many other lattice structures may be used for any of the features disclosed with reference to the figures for use as liners, socket features, or the like. FIGS. 15 and 16 shows a few additional lattice structures that are contemplated. Varying the shape and size of the individual members of the lattice structure may influence the softness, rigidity and other properties of the lattice structure. Similarly, changing the materials, using different processes for curing and/or forming the materials, and other considerations may also influence the relative softness, rigidity and other properties of the resulting lattice structure. Further, the use of a solid layer, such as the outer layers 956, 1056 may influence properties of the 3D printed devices 900, 1000. Removing the solid surface may increase softness, whereas increasing the thickness of the outer layer or using both an inner and outer solid surface layer may increase the rigidity of the overall 3D printed device.

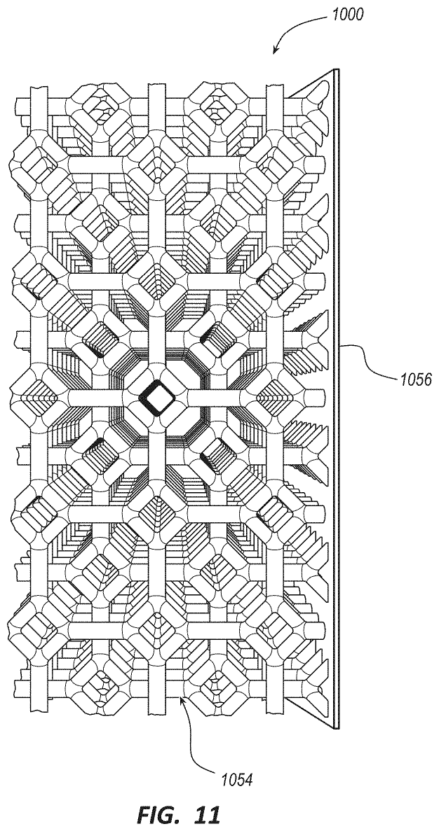

FIG. 12 illustrates another example 3D printed device 1100 that includes a liner portion 1112 and a socket portion 1114. The liner portion 1112 includes one or more liner nubs 1160 positioned around a circumference of the liner portion 1112. The nubs 1160 may extend radially outward from an outer surface of the liner portion 1112. The nubs 1160 may be formed as a continuous structure with the remainder of the liner portion 1112. The nubs 1160 may extend continuously around a perimeter of the liner portion 1112. Alternatively, a plurality of individual nubs may be positioned at spaced apart locations around a circumference and/or along a length of the liner portion 1112.

The socket portion 1114 may include one or more openings 1162 sized to receive one or more of the nubs 1160. The opening 1162 may include separate openings sized to receive each of the individual nubs 1160. In some embodiments, the opening 1162 may be sized and arranged to accommodate multiple nubs 1160. The nubs, when secured within openings 1162 may provide a positive connection between the liner portion 1112 and the socket 1114. The positive connection may limit longitudinal movement of the liner 1112 relative to the socket 1114. One or more openings may not extend completely through the socket portion 1114 and may be recesses on the interior surface of the socket portion. In at least some arrangements, the interface between the nubs 1160 and openings 1162 may limit relative rotational movement between the liner portion 1112 and socket portion 1114. The nubs 1160 may be removed from their position within the openings 1162. For example, a radially inward directed force may be applied to the nubs 1160 to remove them from the openings 1162 such that the liner portion 1112 can be moved relative to the socket portion 1114 (e.g., rotated or translated longitudinally relative to each other).

The liner portion 1112 and socket portion 1114 may comprise lattice structures such as those lattice structures described above with reference to FIGS. 1-11. The lattice structure of the liner portion 1112 may have a relatively soft or compressible construction and may permit fluid flow there through. The socket portion 1114 may comprise a different, more rigid lattice structure that provides additional support for the limb 18.

The liner portion 1112 may be formed separate from the socket portion 1114. The liner portion 1112 and socket portion 1114 may be assembled as shown in FIG. 12 prior to insertion of the limb 18. In other embodiments, the liner 1112 is mounted to the limb 18 followed by the limb 18 with liner 1112 being inserted into the socket portion 1114. The liner portion 1112 and socket portion 1114 may also be formed using various 3D printed methods. The liner portion 1112 and socket portion 1114 may be formed using a 3D model of the limb 18 so as to provide a customized size and shape for an improved interface between the limb 18 and one or both of the liner portion 1112 and socket portion 1114. A 3D model of a limb 18 may be used as part of the manufacturing process for any of the 3D printed devices disclosed herein.

The nub 1160 is shown with a tapered surface along a leading edge that promotes insertion of the nubs 1160 into the internal cavity and openings 1162 of the socket portion 1114 when the liner 1112 is inserted distally into the socket portion 1114. A rear surface of the nubs 1160 have a step surface (e.g., a surface arranged generally perpendicular relative to the outer surface of the liner portion 1112). This step surface may provide an interface with a rear surface of the opening 1162 to limit removal of the liner portion 1112 relative to the socket portion 1114. Other shapes and sizes are possible for the nub 1160 and the openings 1162.

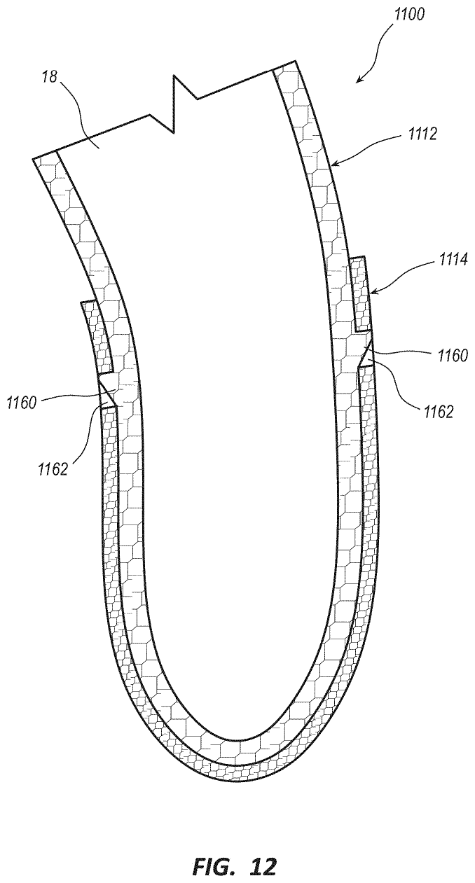

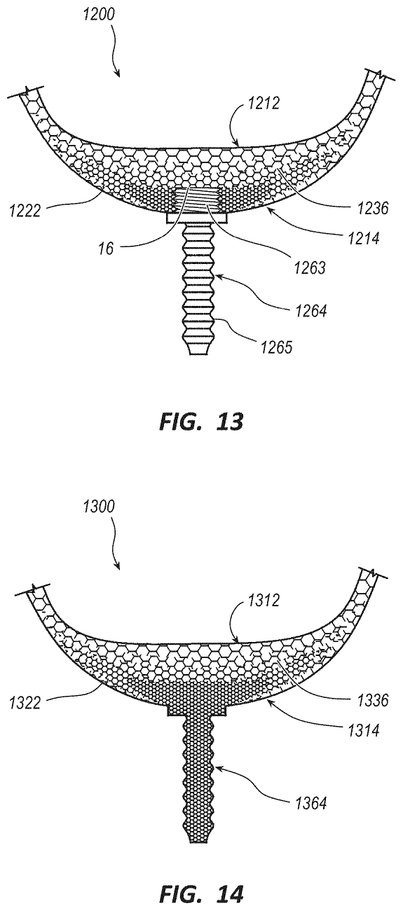

FIGS. 13 and 14 illustrate cross sectional views of distal end portions of additional 3D printed devices having a pin extending distally therefrom. FIG. 13 illustrates a 3D printed device having a relatively soft, first lattice portion 1212, a relatively hard, second lattice portion 1214, and a semi-rigid lattice portion 1236. The semi-rigid portion may be arranged between the soft and hard portions 1212, 1214, or arranged at other locations adjacent one or both of the soft and hard portions 1212, 1214. The 3D printed device 120 may include more than three lattice structures. A receiver 16 may be formed in the hard portion 1214 and configured to releasably connect a pin 1264 to the 3D printed device 1200. The pin 1264 may include a plurality of threads 1263 at a proximal end thereof that are configured to threadibly engage receiver 16. The pin 1264 may also include an engagement portion 1265. The engagement portion 1265 may be configured to releasably secure the 3D printed device 1200 to a prosthetic component such as a prosthetic socket.

The lattice structure of the 3D printed device 1200 may have a variable lattice density through its thickness at the distal end 1222 of the device. The variable density of the lattice may change from a softer or less dense lattice structure 1212 to the semi-rigid lattice structure 1236 to the hard lattice structure 1214, wherein the hard lattice structure is the most dense and/or rigid. The lattice structure of the 3D printed device 1200 may include not only a variation in the density of the lattice structure but also a variation in the structure itself including, for example, the shape and size of individual struts of the lattice structure, the shape and size of the resultant lattice structure (e.g., hexagonal, circular, spherical, etc.), or the relative orientation of the lattice structure members. Furthermore, the 3D printed device 1200 may have different material compositions for the various portions. For example, a harder more rigid material may be used in the area of the hard portion 1214 to provide additional support for the pin 1264.

Generally, the 3D printed device 1200 may be formed as a separate piece from the pin 1264, and the pin 1264 may be mounted and/or assembled to the 3D printed device 1200 in a separate assembly step. In another embodiment, a 3D printed device 1300 shown in FIG. 14 includes a pin 1364 that is integrally formed as a single piece with the remaining soft portion 1312, hard portion 1214 and semi-rigid portion 1236. The pin 1364 may comprise a similar material as other portions of the 3D printed device 1300. For example, the pin 1364 may comprise a polymeric material, which when formed is a solid structure has significant rigidity and strength, and when formed as a lattice structure, such as the soft portion 1212, has a relatively soft, compressible and resilient structure.

In other embodiments, the pin 1364 may be formed as an integral piece with the hard portion 1314 and/or other portions of the 3D printed device 1300. Remaining portions of the 3D printed device 1300 are formed directly onto or into integral connection with the preformed pin 1364 and/or hard portion 1314 such that the entire 3D printed device 1300 is considered an integral single piece. Other manufacturing methods and techniques may be used to form the 3D printed devices 1200, 1300 described with reference to FIGS. 13 and 14.

FIG. 15 illustrates atomic cell structures used as fundamental building blocks for many of the lattice structures that could be used with the 3D printed devices disclosed herein. The cell structures show in FIG. 15 can mimic naturally occurring atomic structures such as cubic, tetragonal, orthorhombic, rhombohedral, monoclinic, triclinic, including body centered, face centered, and base centered variations of these atomic cells

FIGS. 16A-16F illustrate various lattice structures that may be possible for use with the 3D printed devices disclosed herein. Each of the lattice structures 1570 (FIG. 16A), 1572 (FIG. 16B), 1574 (FIG. 16C), 1576 (FIG. 16D), 1578 (FIG. 16E), and 1580 (FIG. 16F) have unique shapes, sizes, and orientations for the individual strut members of the lattice structure as well as the resulting shapes and other features of the lattice structure as a whole. The various lattice structures shown in FIGS. 16A-16F may each provide different properties such as strength, compressibility, flexibility, elasticity, resistance to torque, etc. FIG. 16F shows an example of a lattice unit cell utilizing both semi-circular and straight beams between unit cell connection points. Because the curved beams are not as stiff as the straight beams, the cell is stiffer in compression in Direction 3 than and in Directions 1 and 2. When the straight beams are compressed in Direction 1, the straight beams will demonstrate a high stiffness until buckling occurs, at which point the stiffness will decrease dramatically. The behavior of the curved beams, when compressed in either Direction 1 or 2, do not exhibit buckling behavior. The curved beams are pre-buckled by their semi-circular shape.

The examples shown in FIG. 16A-16F are exemplary only of the infinite number of lattice structure designs that are possible. The lattice designs that are used for various portions of any of the 3D printed devices disclosed herein may be optimized for use as liner, socket, connector pin, internal liner surfaces, exterior protective surfaces, and other features of a 3D printed device that is used and/or capable of being used with a limb such as a residual limb of an amputee.

The various lattice structures disclosed herein may provide certain advantages as compared to other types of materials such as the ability to integrally form a liner structure with a relatively soft liner structure and a relatively rigid or hard socket structure. The various lattice structures may also provide breathability, heat transfer, and the like that are not available with existing liners and/or sockets for use with residual limbs. Further, the example lattice structures disclosed herein may be easily adjusted in size, shape, orientation, and position along the device in order to customize the interface with a residual limb, provide support, cushioning, and/or flexibility to address certain specific features of the residual limbs such as a joint, termination point of a bone, scar tissue on the limb, or the like.

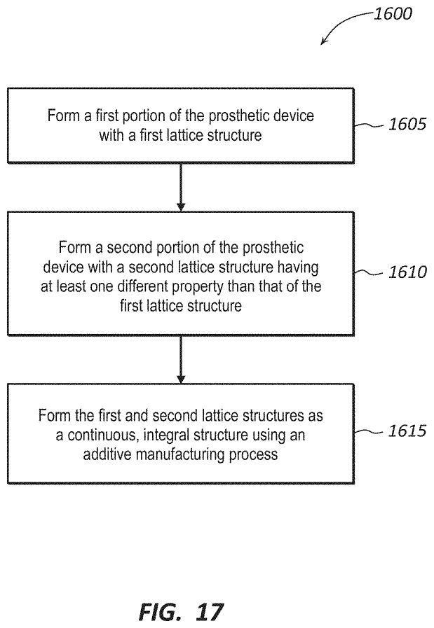

FIG. 17 is a flow diagram illustrating an example method of forming a 3D printed device, particularly a 3D printed device that is configured for use with a person's limb, such as a prosthetic device. A first step of a method 1600 includes forming a first portion of the prosthetic device with a first lattice structure. A second step 1610 may include forming a second portion of the prosthetic device with a second lattice structure having at least one different property than that of the first lattice structure. A step 1615 includes forming the first and second lattice structures as a continuous, integral structure using an additive manufacturing process.

The at least one different property may include at least one of lattice density, material composition, lattice structure, compressibility, porosity, and rigidity. The first portion may be a liner and the second portion may be a socket. The prosthetic device may be a flexible liner, and the first portion is an inner layer of the liner, and the second portion is a second layer of the liner. When the prosthetic device is a socket, the first portion may be a first layer of the socket and the second portion may be a second layer of the socket.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the present systems and methods and their practical applications, to thereby enable others skilled in the art to best utilize the present systems and methods and various embodiments with various modifications as may be suited to the particular use contemplated.

Unless otherwise noted, the terms “a” or “an,” as used in the specification and claims, are to be construed as meaning “at least one of.” In addition, for ease of use, the words “including” and “having,” as used in the specification and claims, are interchangeable with and have the same meaning as the word “comprising.” In addition, the term “based on” as used in the specification and the claims is to be construed as meaning “based at least upon.”

What is claimed is:

1. A prosthetic device, comprising:
a polymer lattice structure comprising:
a first surface arranged to face toward a residual limb;
a second surface arranged to face away from the residual limb;
a thickness between the first and second surfaces;
a variable lattice density across the thickness.

2. The prosthetic device of claim 1, wherein the prosthetic device is arranged to contact a user's skin, and the lattice density increases from the first surface toward the second surface.

3. The prosthetic device of claim 1, wherein the lattice structure has a continuous, single-piece construction.

4. The prosthetic device of claim 1, wherein the lattice structure has a void content of at least 5%.

5. The prosthetic device of claim 1, wherein the lattice structure has a porosity that permits airflow through the thickness from the first surface to the second surface.

6. The prosthetic device of claim 1, wherein the lattice structure comprises an elastomeric material.

7. The prosthetic device of claim 1, wherein the lattice structure comprises an antimicrobial material.

8. The prosthetic device of claim 1, further comprising a fabric material positioned on the first surface, the lattice structure being formed directly on the fabric material.

9. The prosthetic device of claim 1, wherein the prosthetic device comprises a prosthetic sleeve or an orthotic liner.

10. A prosthetic liner, comprising:
a polymer lattice structure comprising:
a first surface arranged to face toward a residual limb;
a second surface arranged to face away from the residual limb;
a porosity that permits airflow through the lattice structure from the first surface to the second surface;
a closed distal end;
an open proximal end.

11. The prosthetic liner of claim 10, wherein the lattice structure includes a thickness between the first and second surfaces, and a variable lattice density across the thickness.

12. The prosthetic liner of claim 11, wherein the lattice density is lowest at the first surface and highest at the second surface.

13. The prosthetic liner of claim 10, wherein the lattice structure comprises an elastomeric material.

14. The prosthetic liner of claim 10, further comprising a receiver formed in the closed distal end, the receiver configured to connect the liner to a prosthetic device.

15. A prosthetic socket, comprising:
a first surface arranged to face toward a residual limb, the first surface having a first rigidity;
a second surface arranged to face away from the residual limb, the second surface having a second rigidity that is greater than the first rigidity;
a continuous, single-piece construction.

16. The prosthetic socket of claim 15, wherein the first surface includes a first structural density and the second surface includes a second structural density.

17. The prosthetic socket of claim 16, further comprising a plurality of apertures formed therein and extending through at least the second surface.

18. The prosthetic socket of claim 15, further comprising a closed distal end, an open proximal end, a hollow interior sized to receive the residual limb, and connection feature formed in the closed distal end.

19. A method of manufacturing a prosthetic device, comprising:
forming a first portion of the prosthetic device with a first lattice structure;
forming a second portion of the prosthetic device with a second lattice structure having at least one of a different property than that of the first lattice structure;
wherein the first and second lattice structures are formed as a continuous, integral structure using an additive manufacturing process.

20. The method of claim 19, wherein the at least one different property includes at least one of lattice density, material composition, lattice structure, compressibility, porosity and rigidity.

21. The method of claim 19, wherein the first portion is a liner and the second portion is a socket.

22. The method of claim 19, wherein the prosthetic device is a flexible liner, the first portion is an inner layer of the liner, and the second portion is a second layer of the liner.

23. The method of claim 19, wherein the prosthetic device is a socket, the first portion is a first layer of the socket, and the second portion is a second layer of the socket.
